(19) United States
(12) Patent Application Publication
(10) Pub. No.: US 2011/0243897 A1
(43) Pub. Date: Oct. 6, 2011

(54) MODIFICATION OF NUCLEIC ACID VECTORS WITH POLYMERS COMPRISING CHARGED QUATERNARY AMINO GROUPS

(76) Inventors: Leonard William Seymour, Oxford (GB); Karel Ulbrich, Prague (CZ)

(21) Appl. No.: 13/139,151

(22) PCT Filed: Dec. 11, 2008
(86) PCT No.: PCT/GB08/04097

§ 371 Date: Jun. 10, 2011

(51) Int. Cl. A61K 48/00; C12N 7/06; A61P 37/04

(52) U.S. Cl. 424/93.6; 435/238

(57) ABSTRACT

The present invention provides a polymer modified nucleic acid vector in which the nucleic acid vector is covalently linked to a polymer, which polymer comprises one or more positively charged quaternary amino groups.

The present invention relates to improved methods of modifying the biological and/or physico-chemical properties of particulate vectors preferably for delivery of therapeutic transgenes or activities, including viruses, fragments of viruses and self-assembling synthetic vectors. The invention also relates to nucleic acid vectors that have been modified to bring about a modification or change in their biological and/or physico-chemical properties in accordance with the invention, processes for their preparation and their use in various biotechnology strategies in various fields, including medicine.

### BACKGROUND

Micro-organisms, including viruses, find many applications throughout the broad fields of biotechnology. They are involved in medicine, agriculture, industrial production processes (including notably the oil and brewing industries) and bioremediation. Many useful applications and functions have been identified and developed for such biological agents. However, often the development or enhancement of their activities is limited by their precise properties, restricting their ability to fulfil tasks that are theoretically possible but practically beyond their scope. In this situation, which is quite commonly encountered, it would often be desirable to re-engineer the properties of the virus or micro-organism to endow it with properties more appropriate for its required purpose.

Thus, biological insecticides for example such as baculoviruses may be restricted in their usefulness through inappropriate target specificity and adverse survival characteristics in the environment; sulphur metabolising bacteria may be limited in their useful application in the petrochemical industry through inadequate patterns of dispersion and distribution; and in the context of human or veterinary gene therapy, viruses intended to mediate delivery of therapeutic genes may be limited in their usefulness through inefficiency of transgene expression in target tissues.

The field of somatic cell gene therapy has attracted major interest in recent years because it promises to improve treatment for many different types of disease, including both genetic diseases (e.g. cystic fibrosis, muscular dystrophy, enzyme deficiencies) and diseases resulting from age- or damage-related physiological deterioration (cancer, heart disease, mature onset diabetes). However, although the field has seen rapid and extensive development, including initiation of over 100 clinical trials, instances of clear therapeutic benefit to patients are very few. One antisense technology has recently been licensed for human use, but no gene therapy strategies have as yet fulfilled their original promise and none are likely to be approved for routine clinical application in the foreseeable future.

A related technological field is known as ‘virotherapy’, where lytic viruses (which may or may not be engineered to carry a therapeutic gene) are able to replicate selectively within cancer cells, leading to amplification of the virus within the tumour, lysis of tumour cells and spread of infection to adjacent tumours cells where the lytic replication cycle is repeated.

The reasons for lack of therapeutic efficacy partly reflect the patient population (most patients enrolled for these experimental treatments are already quite sick so that even an effective treatment might show little therapeutic benefit) but primarily reflect the inadequate levels, duration and distribution of expression of therapeutic genes achieved. In short, the successful application of sophisticated treatment strategies is limited by inadequate vectors for gene delivery and expression.

Two main types of vectors for use in gene therapy applications have been explored so far: non-viral (usually based on cationic liposomes) and viral (usually retroviruses, adenoviruses, latterly adeno-associated viruses (aav) and lentiviruses).

Lytic viruses developed for virotherapy include adenoviruses (all serotypes), herpes virus, toga viruses (notably alpha viruses such as Sindbis and Semliki Forest Virus), cardio virus (notably Seneca Valley Virus), vaccinia virus, Vesiculo Stomatitis Virus, Newcastle Disease Virus, measles virus and reovirus.

Viruses are the obvious choice as vectors for gene delivery since this is essentially their sole function in nature. Consequently viruses have seen considerable use in gene therapy to date, forming the majority of vectors employed in clinical studies. The main feature of adenoviruses which limits their successful application is their immunogenicity. Although they are professional pathogens, evolved over millions of years as highly efficient gene delivery vectors, their hosts have similarly developed very effective protection mechanisms. Serum and ascites fluid from cancer patients contain antibodies that can completely prevent viral infection in vitro even at high dilution.

Typical human protocols involving adenovirus lead to significant inflammatory responses, as well as inefficient infection of target cells.

Although the non-viral systems have a much better safety record, and are easier to produce in large quantities, they have low specific transfection activity. Efficiency of gene expression in target tissues is also a major problem associated with non-viral systems.

Another major limitation to successful application of presently available vectors for treatment of disease is a requirement for their administration directly to the site of disease, either by direct application or by intra-arterial administration. No vectors are capable of targeting to specific cells following intravenous injection. Cationic lipid systems occlude the first capillary bed they encounter, the pulmonary bed, while adenoviruses/retroviruses are rapidly taken up by the liver and (in animal studies) mediate local toxicities. Although local administration can be feasible for treatment of certain diseases (e.g. bronchial epithelial cystic fibrosis), other diseases have a more widespread distribution (notably clinical cancer and atherosclerosis) and intravenous targeted gene delivery is crucial to embrace the possibility of successful gene therapy.

Gene delivery vectors such as DNA-based polyelectrolyte complexes or polymer-modified viruses have been developed to overcome these problems.

One approach described in WO 98/44143 for facilitating clinical use of viruses has been to modify the surface of the viruses with a mono-functional polymer such as poly (ethylene-glycol) (PEG) bearing a terminal amine-reactive group. This can lead to decreased neutralisation of infection by serum antibodies. This approach retains normal receptor-binding and infection in respect of target cells (via the CAR receptor for type 5 adenovirus), but presents a problem in that it does not mediate ablation of normal infectivity (to remove unwanted infection of non-target cells) nor facilitate re-targeting of the virus to selected receptors to gain useful and therapeutically-relevant tropisms.

WO 00/74722 describes a method of modifying the biological and/or physicochemical properties of a biological element such as a virus by providing it with a coating of a multivalent polymer having multiple reactive groups. This approach can enable some biological elements to be targeted or re-targeted to particular sites in a host biological system and can be useful in connection with viral vectors for gene therapy or antitumour therapy.

However, these existing methodologies are not able to coat nucleic acid vector completely, nor are they able to selectively coat those areas of the vectors most susceptible to recognition by antibodies.

It has now been surprisingly found that nucleic acid vectors are much more rapidly and efficiently coated when reactive polymers comprising positively charged quaternary amino groups are employed. Further, it has surprisingly been found that when coated in accordance with the present invention, certain regions of the nucleic acid vectors may be masked that would otherwise be subject to recognition by antibodies. Such regions are typically negatively charged or acid regions on the surface of the nucleic acid vector.

### SUMMARY OF THE INVENTION

The present invention therefore provides a polymer modified nucleic acid vector in which the nucleic acid vector is covalently linked to a polymer, which polymer comprises one or more positively charged quaternary amino groups.

Also provided is a process for modifying the biological and/or physicochemical properties of a nucleic acid vector, said method comprising reacting said nucleic acid vector with a polymerwhich polymer comprises one or more positively charged quaternary amino groups and one or more reactive groups, so that the nucleic acid vector is linked to the polymer by one or more covalent linkages to obtain a polymer modified nucleic acid vector.

Also provided is a polymer-modified nucleic acid vector obtainable by the process of the present invention.

Also provided is a composition comprising a polymer-modified nucleic acid vector of the invention in association with a suitable diluent or carrier.

Also provided is a method of gene therapy (including genetic vaccination) which method comprises administering to a patient in need of such therapy a therapeutically active, non-toxic amount of a polymer-modified nucleic acid vector of the invention or a composition of the invention, which polymer-modified nucleic acid vector comprises therapeutic genetic material.

Also provided is use of a polymer-modified nucleic acid vector of the invention in the manufacture of a medicament for use in vaccination or gene therapy, wherein the polymer-modified nucleic acid vector comprises therapeutic genetic material.

### DETAILED DESCRIPTION OF THE INVENTION

As used herein, the term “nucleic acid vector” refers to a vehicle comprising nucleic acid. Typically, the nucleic acid vector includes therapeutic genetic material.

It will be understood that the term “therapeutic genetic material” is used herein to denote broadly any genetic material or nucleic acid administered for obtaining a therapeutic effect, e.g. by expression of therapeutically useful proteins or RNAs.

It will be appreciated that in the polymer modified nucleic acid vector of the invention, usually no unreacted reactive groups will be present. However, there may be circumstances where some unreacted reactive groups remain in the polymer modified nucleic acid vector, so that biologically active agents may be introduced, for example. In those circumstances, therefore, the polymer modified nucleic acid vector further comprises one or more reactive groups.

Generally, the linkage of the polymers to the nucleic acid vector and modification of the latter results in the inhibition of the ability of the nucleic acid vector to interact in a host biological system with other molecules with which they would otherwise normally interact or in the inhibition of the ability of the nucleic acid vectors to bind to sites or receptors to which they would otherwise normally bind. Certain desirable interactions of the nucleic acid vectors will, of course, remain. The linkage of the polymers to the nucleic acid vector typically results in the inhibition of the ability of the nucleic acid vector to interact with molecules such as serum antibodies that would normally neutralise the nucleic acid vector.

Typically, the nucleic acid vector is a micro-organism chosen from the group consisting of a virus, a bacteria, a bacteriophage, a fungus, a spore, a eukaryotic cell nucleus or other micro-organism fragment or a component containing genetic information.

Viruses and viral particles are preferred. More preferably, the nucleic acid vector is a viral vector containing therapeutic genetic material or a virus with intrinsic therapeutic activity. In principle any known virus may be used in the present invention as the nucleic acid vector. The virus is preferably a recombinant genetically engineered virus. The recombinant virus optionally contains a transgene. It will be understood that the term “transgene” is used herein to denote a nucleic acid which is not native to a virus. For example, a transgene could encode a biologically functional protein or peptide, an antisense molecule, or a marker molecule. The virus is either an RNA or DNA virus and is optionally from one of the following families and groups: Adenoviridae; Alfamoviruses; Bromoviridae; Alphacryptoviruses; Partitiviridae; Baculoviridae; Badnaviruses; Betacryptoviruses; Partitiviridae; Bigeminiviruses; Geminiviridae; Birnaviridae; Bromoviruses; Bromoviridae; Bymoviruses; Potyviridae; Bunyaviridae; Caliciviridae; Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus CD6 phage group; Cystoviridae; Cytorhabdoviruses; Rhabdoviridae; Group Carnation ringspot; Dianthovirus virus group; Group Broad bean wilt; Enamoviruse; Fabavirus virus group; Fijiviruses: Reoviridae; Filoviridae; Flaviviridae; Furovirus group; Group Geminivirus; Group Giardiavirus; Hepadnaviridae; Herpesviridae; Hordeivirus virus group; Hybrigeminiviruses: Geminivirida; Idaeoviruses; Ilarvirus virus group; Inoviridae; Ipomoviruses; Potyviridae; Iriodoviridae; Levivridae; Lipothrixviridae; Luteovirus group; Machlomoviruses; Macluraviruses; Marafivirus virus group; Maize chlorotic dwarf virus group; icroviridae; Monogeminiviruses: Geminiviridae; Myoviridae; Nanaviruses; Necrovirus group; Nepovirus virus group; Nodaviridae; Nucleorhabdoviruses; Rhabdoviridae; Orthomyxoviridae; Oryzaviruses: Reoviridae; Ourmiaviruses; Papovaviridae; Paramyxoviridae; Parsnip yellow fleck virus group; Partitiviridae; Parvoviridae including adeno associated viruses; Pea enation mosaic virus group; Phycodnaviridae; Phytoreoviruses: Reoviridae; Picornaviridae; Plasmarviridae; Podoviridae; Polydnaviridae; Potexvirus group; Potyvirus; Poxyiridae; Reoviridae; Retroviridae; Rhabdoviridae; Group Rhizidiovirus; Rymoviruses: Potyviridae; Satellite RNAs; Satelliviruses; Sequiviruses: Sequiviridae; Sobemoviruses; Siphoviridae; Sobemovirus group; SSVI-Type Phages; Tectirividae; Tenuivirus; Tetravirirdae; Group Tobamovirus; Group Tobravirus; Togaviridae; Group Tombusvirus; Tospoviruses; Bunyaviridae; Group Torovirus; Totiviridae; Tymoviruses; Group Tymovirus; Plant virus satellites; Umbraviruses; Unassigned potyviruses; Potyviridae; Unassigned rhabdoviruses; Rhabdoviridae; Varicosaviruses; Waikaviruses; Sequiviridae; Ungrouped viruses.

Generally, the nucleic acid vector is a virus that normally interacts with particular sites or receptors in a host, wherein the monovalent or multivalent reactive polymer bearing positively charged quaternary amino groups masks the normal receptor-binding activity of the virus and/or enables retargeting of it to a new or different site or receptor in the host.

The nucleic acid vector may be a retrovirus, adenovirus, adenoassociated virus, baculovirus, herpesvirus, papovavirus or poxvirus. For some applications, the nucleic acid vector may be a recombinant virus based on adenovirus, herpes virus, vaccinia virus or alpha virus.

Preferably, the nucleic acid vector is a virus based on adenovirus, herpes virus, parvovirus, poxvirus, Togavirus, Rotavirus or picornavirus.

Adenovirus is especially preferred. Adenoviruses include non-human adenoviruses such as avian adenovirus CELO.

In some cases where the nucleic acid vector is a virus having an outer envelope a preliminary step before reacting it with the polymer may comprise stripping off the envelope.

A component of a nucleic acid vector which is suitable for use as the nucleic acid vector may be provided by, for example, a viral core or a provirus (from e.g. pox viruses). An example of a viral core is an adenovirus core which is preparable by the method disclosed in Russell, W. C., M., K., Skehel, J. J. (1972). “The preparation and properties of adenovirus cores” Journal of General Virology 11, 35-46 and modifications thereto.

Typically, the nucleic acid vector is a virus or viral core.

Bacterial nucleic acid vectors used in carrying out the invention may include, for example, bacteria used in experimental gene therapy (e.g. salmonella), bacteria or baculovirus used as a biological pesticide (e.g. nuclear polydedrosis virus NPD, nonocclude virus NV, granulosis virus or bacillus thuringiensis), a bacteria strain useful for degrading oil sludges/spills or a genetically modified version thereof (e.g. enterobacteriaceae, anitratum, pseudomonas, micrococcus, comamonas, zanthomonas, achromobacter or vidrio-aeromonas), a bacterial strain responsible for reducing sulphur to H2S in oil (e.g. petrotoga snobilis, petrotoga miotherma, desulfotomaculum nigrif cans, desulphovibrio) or a bacterial strain capable of oxidising sulphur from oil (e.g. rhodococcus sp. Strain ECRD-1).

A further example of a bacterium which is suitable for use as the nucleic acid vector is one selected from the group consisting of Rickettsiella popiliae, Bacillus popiliae, B. thuringiensis Including its subspecies israelensis, kurstaki and B. sphaericus), B. lentimorbus, B. sphaericus, Clostridium malacosome, Pseudomonas aeruginosa and Xenorhabdus nematophilus.

A phage which is suitable for use as the nucleic acid vector is for example one from one of the following families: Cyanophages, Lambdoid phages, Inovirus, Leviviridae, Styloviridae, Microviridae, Plectrovirus, Plasmaviridae, Corticoviridae, Satellite bacteriophage. Myoviridae, Podoviridae, T-even phages. An example of a particular phage is MV-L3, PI, P2, P22, d) 29, SPOI, T4, T7, MV-L2, PM2, F1, MV-L51, ou174,06, MS2, M13, Qp, tectiviridae (eg. PRD1).

A fungus which is suitable for use as the nucleic acid vector is for example one from family Basidiomycetes (which make basidiospores. which include classes such as Gasteromycetes, hymenomycetes. urediniomycetes, ustilaginomycetes), Beauveria, Vetarrhizium, Entomophthora or Coelomomyces. A spore which is suitable for use as the nucleic acid vector is a basidiospore, actinomyceres, arthrobacter, microbacterium, clostridium, Rhodococcus, Thermomonospora or Aspergillus fumigatus.

A further example of a bacterium which is suitable for use as the nucleic acid vector is one selected from the group consisting of Rickettsiella popiliae, Bacillus popiliae, B. thuringiensis dIncluding its subspecies israelensis, kurstaki and B. sphaericus), B. lentimorbus, B. sphaericus, Clostridium malacosome, Pseudomonas aeruginosa and Xenorhabdus nematophilus.

In one embodiment, the nucleic acid vector is formed by self assembly between a nucleic acid and a positively charged polymer and/or lipid. The term nucleic acid includes synthetic molecules such as siRNA, antisense RNA and antisense DNA. Usually, the nucleic acid is mRNA or DNA. In this embodiment, the polyelectrolyte vector so formed generally bears a net negative surface charge. Thus, the charge ration (+/−) of the nucleic acid and positively charged lipid or polymer is typically less than 1.0. A charge ratio of 0.8-0.9 is preferred.

Typically, the polymer present in the polymer modified nucleic acid vector of the invention is a multivalent polymer.

Typically, the polymer is linked to the nucleic acid vector by two or more linkages, the polymer used being a multivalent polymer, i.e. it includes multiple reactive groups. The number of linkages between the polymer and the nucleic acid vector is preferably three or more, more preferably four or more. The number of linkages may be, for example, 12 or 14. The advantage of having a higher number of linkages is that the polymer modified nucleic acid vector is more stable.

The polymer backbone is preferably based upon monomer units such as N-2-hydroxypropylmethacrylamide (HPMA), N-(2-hydroxyethyl)-1-glutamine (HEG), ethyleneglycol-oligopeptide or is a polysialic acid or polymannan polymer. HPMA is preferred. Where the backbone is based upon ethyleneglycol-oligopeptide, the oligopeptide group preferably comprises from 1 to 4 peptide groups.

Typically the polymers used in the present invention are prepared using living radical polymerisation methods, such as ATRP (Atom Transfer Radical Polymerisation) or RAFT (Reversible addition-fragmentation chain transfer), for example as described in Scales, C. W.; Vasilieva, Y. A.; Convertine, A. J.; Lowe, A. B.; McCormick, C. L. Biomacromolecules 2005, 6, 1846-1850; Yanjarappa, M. J.; Gujraty, K. V.; Joshi, A.; Saraph, A.; Kane, R. S. Biomacromolecules 2006, 7, 1665-1670; Convertine, A. J.; Ayres, N.; Scales, C. W.; Lowe, A. B.; McCormick, C. L. Biomacromolecules 2004, 5, 1177-1180, the entirety of which are incorporated herein by reference.

Relevant teaching can also be found in ‘Macromolecular design via reversible addition-fragmentation chain transfer (RAFT)/xanthates (MADLY) polymerization.’ Perrier, Sebastien; Takolpuckdee, Pittaya. J. Polym. Sci., Part A: Polym. Chem. (2005), 43(22), 5347-5393, which is incorporated herein by reference.

Typically, the polymer and/or the linkages between it and the nucleic acid vector are hydrolytically or enzymatically degradable.

Instability provided by hydrolytic degradability can be desirable since it permits regulation of the time for which the nucleic acid vector is protected. Thus, if the polymer is provided with a tissue-specific targeting group, i.e. a biologically active agent as defined herein, the polymer (or the linkage between the polymer and the nucleic acid vector) can be designed so that the polymer protects the nucleic acid vector for as long as it takes for the modified nucleic acid vector to reach the appropriate location within the target tissue before disintegrating, freeing the nucleic acid vector to interact with the tissue. Alternatively, the polymer could be designed to disintegrate at a rate yielding optimal kinetics of release of the nucleic acid vector.

Instability provided by enzymatic degradability can be desirable since it permits the polymer (or the linkage between the polymer and the nucleic acid vector) to be designed for cleavage selectively by chosen enzymes. Such enzymes could be present at the target site, endowing the modified nucleic acid vector with the possibility of triggered disintegration at the target site, thereby releasing the nucleic acid vector for interaction with the target tissue. The enzymes may also be intracellular enzymes which can bring about disintegration of the modified nucleic acid vector in selected cellular compartments of a target cell to enhance the activity of the nucleic acid vector. Alternatively, enzyme-cleavage sites may be designed to promote disintegration of the modified nucleic acid vector in response to appropriate biological activity (eg. arrival of an invading or metastatic tumour cell expressing metalloproteinase). In a further variation, enzymes capable of activating the modified nucleic acid vector may be administered at the appropriate time or site to mediate required disintegration of the modified nucleic acid vector and subsequent interaction of the nucleic acid vector with the tissue.

The polymer used to modify the nucleic acid vector in at least some embodiments is preferably cross-linked such that it forms a hydrogel. The hydrogel is preferably hydrolytically unstable or is degradable by an enzyme, for example matrix metalloproteinases 2 or 9. This is in order that the nucleic acid vectors are immobilised within the hydrogel and so that the release of the nucleic acid vectors can be regulated. Thus, according to one preferred feature of the invention, the process of the invention is carried out under conditions likely to promote crosslinking and hydrogel formation (for example high concentrations of reagents with none present in excess) or in the presence of agents such as diamines likely to promote crosslinking. Formation of hydrogels containing modified nucleic acid vectors would generally be performed using the chemical approaches described in Subr, V., Duncan, R. and Kopeck, J. (1990) “Release of macromolecules and daunomycin from hydrophilic gels containing enzymatically degradable bonds”, J. Biomater. Sci. Polymer Edn., 1 (4) 61-275.

The polymer used in the present invention typically comprises one or more positively charged quaternary amino groups in the polymer backbone or in side chains, preferably in side chains.

Generally, each of the one or more positively charged quaternary amino groups is connected to the polymer backbone either directly or via a spacer group. Typically, the spacer groups are as defined herein. In one embodiment, said spacer group is a group L as defined herein.

The number of positively charged quaternary amino groups in the polymer is preferably such as to provide from 0.25 to 10 mol %, more preferably from 0.5 to 7.5 mol %, and most preferably from 1.5 to 5 mol % of positively charged quaternary amino groups based on the total weight of the polymer.

Usually, the positively charged quaternary amino groups are randomly spaced within the polymer.

When the polymer is a monovalent polymer, i.e. has only one reactive group, the positively charged quaternary amino groups are preferably located near to the reactive group.

Typically, the positively charged quaternary amino groups connected to the polymer backbone are chosen from positively charged quaternary amino groups of formula Ia, Ib, Ic, Id and Ie as defined herein. Positively charged quaternary amino groups of formula Ia are preferred.

Usually, each of the positively charged quaternary amino groups is linked to the polymer via one or more degradable or biodegradable linkages, preferably one degradable or biodegradable linkage. These linkages may either be the direct bond between the positively charged quaternary amino groups and the polymer backbone or, alternatively, a bond in said spacer group. Typically, said linkages refer to reducible, hydrolysable or otherwise cleavable bonds. Examples of such bonds include disulphide bonds, hydrazide bonds, acetal moieties or bonds that are enzymatically cleavable.

Disulphide bonds, —S—S—, are typically cleaved using mild reducing conditions, such as a metal sulphite, or using a suitably chosen enzyme, for example thioredoxin. Typically, cleavage conditions are chosen so that the viability of the vector is unaffected.

Hydrazide bonds, —N—N—, are typically cleaved using mild oxidative or reducing conditions. Again, cleavage conditions are typically chosen so that the viability of the vector is unaffected.

Acetal moieties are well known to the skilled person and are readily cleaved using aqueous acid.

Enzymatically cleavable bonds are typically as discussed herein in relation to the linkages between the reactive groups and the polymer backbone.

In some embodiments, both the nucleic acid vector and the positively charged quaternary amino groups are linked to the polymer via degradable bonds. In this embodiment, the bonds between the nucleic acid vector and the polymer and the bonds between the positively charged quaternary amino groups and the polymer may be cleaved under the same conditions. It is, however, preferred that the bonds between the nucleic acid vector and the polymer and the bonds between the positively charged quaternary amino groups and the polymer are not cleaved under the same conditions. Thus, it is possible to cleave the bonds between the positively charged quaternary amino groups and the polymer whilst leaving the bonds between the nucleic acid vectorand the polymer backbone intact. In this way, the positively charged quaternary amino groups may be removed from the polymer modified nucleic acid vector, leaving the polymers attached to the nucleic acid vector.

It has been found that normally infective nucleic acid vectors such as viruses modified in accordance with the invention lose their original infectivity. Infectivity may be restored or replaced, however, in certain preferred embodiments by coupling a biologically active agent to the polymer. The biologically active agent is optionally coupled to the polymer either before it is combined with the nucleic acid vector or after. Preferably, in cases where the targeting agent, i.e. biologically active agent, has a plurality of reactive groups it is coupled to the polymer after the polymer has coated the nucleic acid vector to avoid it interfering with the coupling reaction, but in other cases it may be satisfactory to couple it to the polymer before coating the nucleic acid vector. Typically, a biologically active agent is coupled to or included in the polymer.

The biologically active agent may be incorporated using the same type of reactive groups as are used to couple the reactive polymer to the nucleic acid vector, or it may be coupled using different chemistry. In the latter situation, a heteromultifunctional reactive polymer (for example containing mixed ONp esters and thiol groups) would be used.

Such biologically active agents are preferably incorporated in accordance with the invention to improve targeting, tissue penetration, pharmacokinetics or immune stimulation or suppression. The biologically active agent may be, for example, a growth factor or cytokine, a sugar, a hormone, a lipid, a phospholipid, a fat, an apolipoprotein, a cell adhesion promoter, an enzyme, a toxin, a peptide, a glycoprotein, a serum protein, a vitamin, a mineral, an adjuvant molecule, a nucleic acid, an immunomodulatory element or an antibody recognising a receptor, for example a growth factor receptor or recognising a tissue-specific antigen or tumour-associated antigen. The agent may be Sialyl Lewis X which can be used to target endothelial tissue.

An antibody is preferably used as the biologically active agent to re-target modified nucleic acid vectors to a different target site which may comprise, for example, various receptors, different cells, extracellular environments and other proteins. A wide range of different forms of antibody may be used including monoclonal antibodies, polyclonal antibodies, diabodies, chimeric antibodies, humanised antibodies, bi-specific antibodies, camalid antibodies, Fab fragments, Fc fragments and Fv molecules.

For use in targeting tumours a suitable biologically active agent is for example an antibody recognizing a cancer associated antigen such as a carcinoembryonic antigen or α-fetoprotein, tenascin, HER-2 proto-oncogene, prostate specific antigen or MUC-1 or an antibody recognising an antigen associated with tumour-associated endothelial cells, such as receptors for vascular endothelial growth factor (VEGF), Tie1, Tie2, P-selectin, E-selectin or prostate-specific membrane antigen (PSMA).

A suitable multi-purpose protein for use as the biologically active agent to act as a generic linker permitting flexibility of application is protein G (this will bind an antibody, allowing surface modification with any IgG class antibody from most species), protein A (which has properties similar to protein G), avidin (which binds biotin with very high affinity allowing the incorporation of any biotin labelled element onto the surface), streptavidin (which has properties similar to avidin), extravidin (which has properties similar to avidin), bungaratoxin-binding peptide (which binds to bungaratoxin fusion proteins), wheat germ agglutinin (which binds sugars), hexahistidine (which allows for gentle purification on nickel chelate columns), GST (which allows gentle purification by affinity chromatography).

A suitable growth factor or cytokine for use as the biologically active agent is for example Brain derived neurotrophic factor, Cilary neurotrophic factor, b-Endothelial growth factor, Epidermal growth factor (EGF), Fibroblast growth factor Acidic (aFGF), Fibroblast growth factor Basic (bFGF), Granulocyte colony-stimulating factor, Granulocyte macrophage colony-stimulating factor, Growth hormone releasing hormone, Hepatocyte growth factor, Insulin like growth factor-I, Insulin like growth factor-II, Interleukin-1a, Interleukin-1b, Interleukin 2, Interleukin 3. Interleukin 4, Interleukin 5, Interleukin 6, Interleukin 7, Interleukin 8, Interleukin 9, Interleukin 10, Interleukin 11. Interleukin 12, Interleukin 13. Keratinocyte growth factor, Leptin, Liver cell growth Factor, Macrophage Colony stimulating factor, Macrophage inflammatory protein 1a, Macrophage inflammatory protein 1b, Monocyte chemotactic protein 1,2-methoxyestradiol, b-nerve growth factor, 2.5s nerve growth factor, 7s nerve growth factor, Neurotrophin-3, Neurotrophin-4, Platelet derived growth factor AA, Platelet derived growth factor AB, Platelet derived growth factor BB, Sex hormone binding globulin, Stem cell factor, Transforming growth factor-β1, Transforming growth factor-β3, Tumour necrosis factor α, Tumour necrosis factor β, Vascular endothelial growth factor, and Vascular endothelial growth factor C.

A suitable sugar for use as the biologically active agent for incorporation is a monosaccharide, disaccharide or polysaccharide including a branched polysaccharide is, for example, D-Galactose, D-Mannose, D-Glucose, L-Glucose, L-Fucose, and Lactose. Sugars are typically incorporated by amino derivitisation.

A hormone which is suitable for use as the biologically active agent is, for example, Adrenomedullin, Adrenocorticotropic hormone, Chorionic gonadotropic hormone, Corticosterone, Estradiol, Estriol, Follicle stimulating hormone, Gastrin 1, Glucagon, Gonadotrophin, Growth hormone, Hydrocortisone, Insulin, Leptin, Melanocyte stimulating hormone, Melatonin, Oxytocin, Parathyroid hormone, Prolactin, Progesterone, Secretin, Thrombopoetin, Thyrotropin, Thyroid stimulating hormone, and Vasopressin.

A suitable lipid, fat or phospholipid for use as the biologically active agent for targeting the polymer modified nucleic acid vector or for providing steric protection is, for example, Cholesterol, Glycerol, a Glycolipid, a long chain fatty acid, particularly an unsaturated fatty acid e.g. Oleic acid, Platelet activating factor, Sphingomylin, Phosphatidyl choline, or Phosphatidyl serine.

A suitable cell adhesion promoter for use as the biologically active agent can be provided by, for example, Fibronectin, Laminin, Thrombospondin, Vitronectin, polycations, integrins or by oligopeptide sequences binding integrins or tetraspan proteins.

A suitable apoliproprotein for use as the biologically active agent that may also provide steric protection is, for example, a high-density lipoprotein or a low-density lipoprotein, or a component thereof.

A suitable enzyme for use as the biologically active agent, for example, to promote mobility of the modified nucleic acid vector through a particular environment is an enzyme capable of degrading the extracellular matrix (for example a gelatinase, e.g. matrix metalloproteases type 1 to 11, or a hyaluronidase), an enzyme capable of degrading nucleic acids (for example Deoxyribonuclease I, Deoxyribonuclease Nuclease, Ribonuclease A), an enzyme capable of degrading protein (for example Carboxypeptidase, plasmin, Cathepsins, Endoproteinase, Pepsin, Proteinase K, Thrombin, Trypsin, Tissue type plasminogen activator or Urokinase type plasminogen activator), an enzyme facilitating detection (for example Luciferase, Peroxidase, b-galactosidase), or other useful enzymes such as Amylase, Endoglycosidase, Endo-b-galactosidase, Galactosidases, Heparinase, HIV reverse transcriptase, b-hydroxybutyrate dehydrogenase, Insulin receptor kinase, Lysozyme, Neuraminidase, Nitric oxide synthase, Protein disulphide isomerase.

A suitable toxin for use as the biologically active agent to bind a receptor or to interact with cell membranes is, for example, Cholera toxin B subunit, Crotoxin B subunit, Dendrotoxin, Ricin B chain.

A suitable peptide for use as the biologically active agent may be provided by, for example, transferrin, Green/blue/yellow fluorescent protein, Adrenomedullin, Amyloid peptide, Angiotensin I, Angiotensin II, Arg-Gly-Asp, Atriopeptin, Endothelin, Fibrinopeptide A, Fibrinopeptide B, Galanin, Gastrin, Glutathione, Laminin, Neuropeptide, Asn-Gly-Arg, Peptides containing integrin binding motifs, targeting peptides identified using phage libraries, peptides containing nuclear localisation sequences and peptides containing mitochondrial homing sequences.

A suitable serum protein for use as the biologically active agent is, for example, Albumin, Complement proteins, Transferrin, Fibrinogen, or Plasminogen.

A suitable vitamin or mineral for use as a biologically active agent is, for example, Vitamin B12, Vitamin B16 or folic acid.

Typically, the modification of the nucleic acid vector has the effect of retargeting the nucleic acid vector to different receptors in a biological host.

It will thus be seen that a polymer modified nucleic acid vector in accordance with the present invention can be synthesised so as to be targeted to a highly specific set of cells, e.g. tumour cells. At the same time, however, it has been found that polymer modified nucleic acid vectors in accordance with the present invention are not generally rendered inactive by neutralising antibodies. This is believed to be because the modified nucleic acid vector is shielded by the polymer. The shielding of the nucleic acid vector by the polymer has been found to have other potential advantages, including increased shelf life and better resistance to low pH. Also, it may be possible to purify such nucleic acid vectors using more aggressive technology than that which is feasible with existing unmodified nucleic acid vectors.

Optionally the polymer can be coupled to a radioisotope in order to allow the detection of the nucleic acid vector e.g. in a biological environment.

In one embodiment, the modification of the nucleic acid vector has the effect of modifying the solubility and dispersal and stability characteristics of the nucleic acid vector within a non-aqueous environment. In this embodiment, the nucleic acid vector is generally a micro-organism having oil degradative activity. Preferably, the nucleic acid vector is a baculovirus particle. In this embodiment, the polymer typically incorporates an oleyl or other hydrophobic group.

Virus particles to be coated must normally be highly purified and free of contaminating proteins or peptides. The coating reaction is normally performed within a pH range of 7.4 to 8.4 with 7.8 to 8.0 being preferable. Any suitable buffer may be used to achieve the desired pH apart from those that may react with the polymer (such as Tris based buffers). The reaction can occur in the presence of physiological salts (150 mM NaCl) concentration and other stabilisers such as Mg2+ or Ca2+ that may be used to stabilise virus preperations. It is not advisable to use sodium azide or other preservatives during the reaction process. At room temperature the polymer coating reaction reaches saturation after 1 hour but a longer duration may be required at lower temperatures.

For retargeting or additional functionality, it possible to add additional biological agents such as antibodies, ligands or peptides during the coating reaction as previously described, see for example Fisher K D, Stallwood Y, Green N K, Ulbrich K, Mautner V, Seymour L W. Polymer-coated adenovirus permits efficient retargeting and evades neutralising antibodies. Gene Ther. March 2001; 8(5):341-348, which is incorporated herein by reference. Alternatively coating may be performed with polymers pre-conjugates to the targeting element, see for example Stevenson M, Hale A B, Hale S J, Green N K, Black G, Fisher K D, Ulbrich K, Fabra A, Seymour L W. Incorporation of a laminin-derived peptide (SIKVAV) on polymer-modified adenovirus permits tumor-specific targeting via alpha6-integrins. Cancer Gene Ther. April 2007; 14(4):335-345 which is incorporated herein by reference.

It will be understood that the term “reactive group” is used herein to denote a group that shows significant chemical reactivity, especially in relation to coupling or linking reactions with complementary reactive groups of other molecules, typically with groups on the surface of the nucleic acid vector.

Typically, the reactive group is a group capable of forming a covalent bond with a group present on the surface of the nucleic acid vector, for example with an amine group, thiol, hydroxy group, aldehyde, ketone, tyrosine residue, carboxylic acid or sugar group. Said group present on the surface of the nucleic acid vector may be introduced by genetic engineering, for example, by engineering an adenovirus to contain cysteine residues bearing free thiols in its fibre molecules. Usually, however, said group present on the surface of the nucleic acid vector is a group that is naturally present.

In one embodiment, the reactive group is capable of forming a covalent bond with an amine group on the surface of the nucleic acid vector. Examples of suitable types of reactive group in this embodiment include acid chlorides, acyl-thiazolidine-2-thiones, maleimides, N-hydroxy-succinimide esters (NHS esters) sulfo-N-hydroxy-succinimide esters (Sulfo-NHS esters), 4-nitrophenol esters, epoxides, 2-imino-2-methoxyethyl-1-thioglycosides, cyanuric chlorides, imidazolyl formates, succinimidyl succinates, succinimidyl glutarates, acyl azides, acyl nitriles, dichlorotriazines, 2,4,5-trichlorophenols, azlactones and chloroformates. Such groups react readily with amines. Acyl-thiazolidine-2-thiones and Sulfo-NHS esters are preferred. Acyl-thiazolidine-2-thiones are preferred due to their high reactivity and relative stability in aqueous solutions.

In another embodiment, the reactive group is capable of forming a covalent bond with a thiol group on the surface of the nucleic acid vector. Examples of suitable types of reactive group in this embodiment include alkyl halides, halo acetamides, and maleimides.

In another embodiment, the reactive group is capable of forming a covalent bond with a hydroxyl group on the surface of the nucleic acid vector. Examples of suitable types of reactive group in this embodiment include chloroformates and acid halides. Alternatively, hydroxyl groups on the surface of the nucleicacid vector can be oxidised with an oxidizing agent, e.g. periodate, followed by reaction with reactive groups that include hydrazines, hydroxylamines or amines.

In another embodiment, the reactive group is capable of forming a covalent bond with a tyrosine residue on the surface of the nucleic acid vecotr. Examples of suitable types of reactive group in this embodiment include sulfonyl chlorides and iodoacetamides.

In another embodiment, the reactive group is capable of forming a covalent bond with an aldehyde or ketone group on the surface of the nucleic acid vector. Examples of suitable types of reactive group in this embodiment include hydrazides, semicarbazides, primary aliphatic amines, aromatic amines and carbohydrazides.

In another embodiment, the reactive group is capable of forming a covalent bond with a carboxylic acid on the surface of the nucleic acid vector. This can be effected by, for example, activating a carboxylic acid using the water soluble carbodiimide, 1-ethyl-3-(3-dimethylaminopropyl)carbodiimide hydrochloride followed by reaction with an amine as reactive group.

In another embodiment, the reactive group is capable of reacting with a sugar on the surface of the nucleic acid vector resulting in the formation of a covalent bond. This can be effected by, for example, enzyme-mediated oxidation of the sugar with galactose oxidase to form an aldehyde followed by reaction with an aldehyde reactive compound such as a hydrazide as reactive group.

The number of reactive groups on the polymer is preferably such as to provide from 0.5 to 10 mol %, more preferably froml to 6 mol %. and most preferably from 2 to 5 mol % of reactive groups based on the total weight of the polymer. In general, the polymer is a biologically inert polymer. The polymer backbone is generally substituted by said reactive groups. Usually, the polymer is a biologically inert polymer having a backbone which is substituted by one or more reactive groups. These reactive groups may be connected to the polymer backbone either directly or via a spacer group. Examples of spacer groups include oligopeptide linkages. Such oligopeptide linkages preferably comprise from 1 to 4 peptide groups, especially 2 or 4. Examples of suitable linkages include -Gly-Gly-, -Glu-Lys-Glu-; and -Gly-Phe-Leu-Gly-. In the case of an ethyleneglycol-oligopeptide polymer, it is the oligopeptide group which is substituted by the reactive group, optionally via a spacer group as defined above. In some embodiments, said spacer is a group L as defined herein.

The polymer used in the present invention is preferably a synthetic hydrophilic polymer containing one or more said reactive groups.

More preferably, the polymer used in the present invention is preferably a synthetic hydrophilic multivalent polymer containing a plurality of said reactive groups.

Examples of suitable polymers for use in the invention are those disclosed in WO 98/19710 and include polyHPMA-GlyPheLeuGly-ONp, polyHPMA GlyPheLeuGly-NHS, polyHPMA-Gly-Gly-ONp, polyHPMA-Gly-Gly-NHS, poly (pEG-oligopeptide (—ONp)), poly (pEG-GluLysGlu (ONp)), pHEG-ONp. pHEG-NHS. The preparation of these compounds is disclosed in WO 98/19710. The entirety of WO 98/19710 is incorporated herein by reference.

In one embodiment of the process of the present invention, each of the positively charged quaternary amino groups is connected to the polymer backbone via one or more degradable or biodegradable linkages, typically by linkers containing reducible or hydrolysable bonds, and said process comprises the additional step of cleaving said degradable or biodegradable linkages between the quaternary positively charged amino groups and the polymer backbone.

Generally, in the polymer-modified nucleic acid vectors of the present invention, the polymer masks regions of the nucleic acid vector that would otherwise be subject to recognition by antibodies that can neutralise the activity of the polymer-modified nucleic acid vectors. Typically, said regions are negatively charged or acid regions on the surface of the nucleic acid vectors.

When the nucleic acid vector is adenovirus, said negatively charged regions are typically negatively charged regions of the adenovirus hexon protein, for example, motif 147-162 (EDEEEEDEDEEEEEEE). Such regions are typically unreactive towards the reactive groups on the polymer and generally repel polymers that are slightly negatively charged, e.g. polymers based on HPMA. Thus, incorporation of positively charged quaternary amino groups in the polymers associates the polymers with these regions electrostatically and minimises any possible repulsive force between these regions and the polymers. This therefore increases the rate of reaction between the vector and the polymer comprising positively charged quaternary amino groups.

The compositions according to the invention are typically suitable for in vitro use or for use in plants or animals. Where the composition is for use in an animal, especially a mammalian animal, the carrier is preferably a pharmaceutically acceptable additive, diluent or excipient. Preferred compositions are free of contamination from micro-organisms and pyrogens.

The polymer modified nucleic acid vectors of the invention may be administered in a variety of dosage forms. Thus, they can be administered orally, for example as aqueous or oily suspensions. The polymer modified nucleic acid vectors of the invention may also be administered parenterally, either subcutaneously, intravenously, intramuscularly, intrasternally, introperitoneally, intradermally, transdermally or by infusion techniques. Intraperitoneal and intradermal administration is preferred. The polymer modified nucleic acid vectors may be administered by inhalation in the form of an aerosol via an inhaler or nebuliser.

The formulations for oral administration, for example, may contain, together with the polymer modified nucleic acid vector, solubilising agents, e.g. cyclodextrins or modified cyclodextrins; diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations.

Liquid dispersions for oral administration may be solutions, syrups, emulsions and suspensions. The solutions may contain solubilising agents e.g. cyclodextrins or modified cyclodextrins. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol; solubilising agents, e.g. cyclodextrins or modified cyclodextrins, and if desired, a suitable amount of lidocaine hydrochloride.

Solutions for intravenous or infusions may contain as carrier, for example, sterile water and solubilising agents, e.g. cyclodextrins or modified cyclodextrins or preferably they may be in the form of sterile, aqueous, isotonic saline solutions.

A therapeutically effective amount of a polymer modified nucleic acid vector of the invention is administered to a patient. In the case of a polymer-modified oncolytic virus for virotherapy, typical doses would contain 10⁷-10¹³ virus particles, depending on the individual virus. The polymer modified nucleic acid vector of the invention is typically administered to the patient in a non-toxic amount.

The polymer modified nucleic acid vectors of the present invention are useful for in vivo delivery of therapeutic genetic material to a patient, in carrying out gene therapy or genetic-vaccination treatment for example, wherein the polymer modified nucleic acid vector is a polymer modified nucleic acid vector in accordance with the invention which includes the therapeutic genetic material.

Gene therapy has applications across the whole field of human disease including, but not limited to, the treatment of cancer (including locally accessible tumour nodules suitable for direct injection, as well as metastatic cancer requiring systemic treatment), Parkinson's disease, X-SCID, Sickle Cell Disease, Lesch-Nyhan syndrome, phenylketonuria (PKU), Huntington's chorea, Duchenne muscular dystrophy, hemophilia, cystic fibrosis, lysosomal storage diseases, cardiovascular diseases and diabetes.

The polymer-modified nucleic acid vectors of the invention may also be used for the delivery of viral vaccines. Vaccination against HIV, tuberculosis, malaria, flu, cancer and other diseases are envisaged. Vaccines may be given in prime boost regimes (i.e. by multiple administrations) or in combination with adjuvants.

The polymer modified nucleic acid vectors of the present invention are useful for in vivo delivery of therapeutic agents to a patient, in carrying out microbial therapy including virotherapy for example, wherein the polymer modified nucleic acid vector is a polymer modified nucleic acid vector in accordance with the invention.

In certain embodiments, the polymer modified nucleic acid vector of the present invention may be used in combination with other medicaments, e.g. other medicaments effective in the treatment of cancer.

The present invention also provides a monovalent or multivalent polymer comprising (a) one or more positively charged quaternary amino groups and (b) one or more reactive groups.

These polymers can be preferred polymers for use in the process of the present invention.

The polymer generally comprises a backbone and side chains. The side chains are attached to the polymer backbone.

In one embodiment, the one or more positively charged quaternary amino groups is one or more positively charged quaternary amino groups of formula Ia:

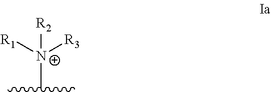

wherein, R₁, R₂ and R₃ are each independently selected from straight or branched C₁-C₆ alkyl groups, straight or branched C₂-C₆ alkenyl groups, straight or branched C₂-C₆ alkynyl groups, 6- to 10-membered aryl groups, 5- to 10-membered heteroaryl groups, C₃-C₈ cycloalkyl groups, and 3- to 8-membered heterocyclyl groups, which C₁-C₆ alkyl groups, C₂-C₆ alkenyl groups, C₂-C₆ alkynyl groups, 6- to 10-membered aryl groups, 5- to 10-membered heteroaryl groups, C₃-C₈ cycloalkyl groups and 3- to 8-membered heterocyclyl groups are unsubstituted or substituted with 1, 2 or 3 substituents selected from halogen atoms, —CN groups, —NH₂ groups, hydroxy groups, —COOH groups, —NO₂ groups, straight or branched unsubstituted C₁-C₄ alkyl groups, straight or branched C₁-C₄ alkoxy groups, straight or branched C₁-C₄ alkylthio groups, straight or branched C₁-C₄ alkylamino groups, 6- to 10-membered aryloxy groups and phenyl groups, which phenyl groups are typically unsubstituted or substituted with 1, 2 or 3 substituents chosen from halogen atoms, —CN groups, —NH₂ groups, hydroxy groups, and —NO₂ groups. Positively charged quaternary amino groups of formula Ia are generally present in sidechains attached to the polymer backbone. Positively charged quaternary amino groups of formula Ia are typically attached to the polymer backbone by a single bond, as shown.

In another embodiment, the one or more positively charged quaternary amino groups is one or more positively charged quaternary amino groups of formula Ib:

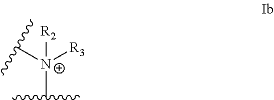

wherein R₂, and R₃. Positively charged quaternary amino groups of formula Ib are generally present in either the polymer backbone or in sidechains attached to the polymer backbone. When present in the sidechain, positively charged quaternary amino groups of formula Ib are typically attached to the polymer backbone by one or two single bonds, as shown, preferably one single bond.

In another embodiment, the one or more positively charged quaternary amino groups is one or more positively charged quaternary amino groups of formula Ic:

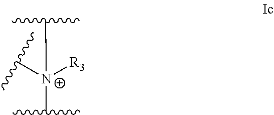

wherein R₃ is as defined above. Positively charged quaternary amino groups of formula Ic are generally present in either the polymer backbone or in sidechains attached to the polymer backbone. When present in the sidechain, positively charged quaternary amino groups of formula Ic are typically attached to the polymer backbone by one, two or three single bonds, as shown, preferably one single bond.

In another embodiment, the one or more positively charged quaternary amino groups is one or more positively charged quaternary amino groups of formula Id:

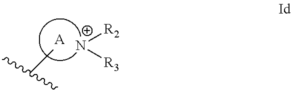

wherein A is a 3- to 8-membered heterocyclyl ring comprising the nitrogen atom to which R₂ and R₃ is bonded, and R₂, and R₃ are as defined above. Ring A in formula Id can optionally contain 1 or 2 heteroatoms chosen from O, S and N in addition to the nitrogen atom to which R₂, R₃ is bonded. Positively charged quaternary amino groups of formula Id are generally present in sidechains attached to the polymer backbone. Positively charged quaternary amino groups of formula Id are typically attached to the polymer backbone by a single bond, as shown.

In another embodiment, the one or more positively charged quaternary amino groups is one or more positively charged quaternary amino groups of formula Ie:

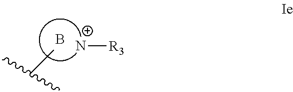

wherein B is a 6- to 10-membered heteroaryl ring comprising the nitrogen atom to which R₃ is bonded, and R₃ is as defined above. Ring B in formula Ie can optionally contain 1 or 2 heteroatoms chosen from O, S and N in addition to the nitrogen atom to which R₃ is bonded. Positively charged quaternary amino groups of formula Ie are generally present in sidechains attached to the polymer backbone. Positively charged quaternary amino groups of formula Ia are typically attached to the polymer backbone by a single bond, as shown.

As used herein, the term C₁-C₆ allyl includes both saturated straight chain and branched alkyl groups. Examples of C₁-C₆ alkyl groups include methyl, ethyl, propyl, isopropyl, butyl, isobutyl, tert-butyl, pentyl and hexyl. Preferably, the C₁-C₆ alkyl group is a C₁₋₄ alkyl group, more preferably a C₁₋₃ alkyl group, even more preferably a methyl or ethyl group, most preferably a methyl group.

As used herein, the term C₂-C₆ alkenyl refers to groups containing one or more carbon-carbon double bonds, which group may be straight or branched. Preferably, the C₂-C₆ alkenyl group is a C₂-C₄ alkenyl group. More preferably, the C₂-C₆ alkenyl group is a vinyl, allyl or crotyl group, most preferably an allyl group.

As used herein, the term C₂-C₆ alkynyl refers to groups containing one or more carbon-carbon triple bonds, which may be straight or branched.

As used herein, the term 6- to 10-membered aryl refers to monocyclic or polycyclic aromatic ring systems such as phenyl or naphthyl. Phenyl is preferred.

As used herein, the term 5- to 10-membered heteroaryl refers to an aromatic ring system comprising at least one heteroaromatic ring and containing at least one heteroatom selected from O, S and N. A heteroaryl group may be a single ring or two or more fused rings wherein at least one ring contains a heteroatom. Examples include pyridyl, pyrazinyl, pyrimidinyl, pyridazinyl, furyl, oxadiazolyl, oxazolyl, imidazolyl, thiazolyl, thiadiazolyl, thienyl, pyrrolyl, pyridinyl, benzothiazolyl, indolyl, indazolyl, purinyl, quinolyl, isoquinolyl, phthalazinyl, naphthyridinyl, quinoxalinyl, quinazolinyl, quinolizinyl, cinnolinyl, triazolyl, indolizinyl, indolinyl, isoindolinyl, isoindolyl, imidazolidinyl, pteridinyl and pyrazolyl radicals. Pyridyl, thienyl, furanyl, pyridazinyl, pyrimidinyl and quinolyl radicals are preferred. Preferably a heteroaryl group is a 5 or 6 membered single ring, for example pyridyl, pyrazinyl, pyrimidinyl, pyridazinyl, furyl, oxadiazolyl, oxazolyl, imidazolyl, thiazolyl, thiadiazolyl, thienyl, pyrrolyl, and pyridinyl.

As used herein, the term C₃-C₈ cycloalkyl group refers to a saturated or unsaturated group. Preferably, the C₃-C₈ cycloalkyl group is saturated. Examples of C₃-C₈ cycloalkyl groups include cyclopropyl, cyclobutyl, cyclopentyl, cyclohexyl, cycloheptyl and cyclooctyl. Preferably, the C₃-C₈ cycloalkyl group is a cyclohexyl group.

As used herein, the term C₃-C₈ heterocyclic group refers to a saturated or unsaturated, non-aromatic, carbocyclic ring, such as a 5, 6 or 7 membered ring, in which one or more, for example 1, 2, or 3 of the carbon atoms, preferably 1 or 2 of the carbon atoms are replaced by a heteroatom selected from N, O and S. Saturated heterocyclic groups are preferred. A heterocyclic group may be a single ring or two or more fused rings wherein at least one ring contains a heteroatom.

Examples of heterocyclic groups include piperidyl, pyrrolidyl, pyrrolinyl, piperazinyl, morpholinyl, thiomorpholinyl, pyrrolyl, pyrazolinyl, pirazolidinyl, quinuclidinyl, triazolyl, pyrazolyl, tetrazolyl, cromanyl, isocromanyl, imidazolidinyl, imidazolyl, oxiranyl, azaridinyl, 4,5-dihydro-oxazolyl and 3-aza-tetrahydrofuranyl.

As used herein, the term halogen atom refers to chlorine, fluorine, bromine or iodine atoms typically a fluorine, chlorine or bromine atom, most preferably chlorine or fluorine. The term halo when used as a prefix has the same meaning.

As used herein, a C₁-C₄ alkoxy group is a said C₁-C₄ alkyl group, for example a C₁-C₂ alkyl group, which is attached to an oxygen atom. Unsubstituted C₁-C₄ alkoxy groups are preferred. Preferably, the C₁-C₄ alkoxy group is a methoxy group.

As used herein, a C₁-C₄ alkylthio group is a said C₁-C₄ alkyl group, for example a C₁-C₂ alkyl group, which is attached to a sulphur atom. Unsubstituted C₁-C₄ alkylthio groups are preferred.

As used herein, a C₁-C₄ alkylamino group is a said C₁-C₄ alkyl group, for example a C₁-C₂ alkyl group, which is attached to a nitrogen atom. Unsubstituted C₁-C₄ alkylamino groups are preferred.

As used herein, the term 6- to 10-membered aryloxy group is a said 6- to 10-membered aryl group, which is attached to an oxygen atom. Unsubstituted phenoxy groups are preferred.

Typically, the polymer comprises one or more positively charged quaternary amino groups selected from groups of formula Ia, Ib, Ic, Id and Ie. Polymers comprising one or more positively charged quaternary amino groups of formula Ia are preferred.

In practice, the polymers are typically the form of a salt with a suitable counterion. Thus, the positive charge on the nitrogen atom is typically associated with an anion A⁻. A⁻ is usually an anion of a mineral acid such as, for example, a halide, e.g. chloride, bromide or iodide, sulphate, nitrate, phosphate, hexafluorophosphate and tetrafluoroborate, or an anion of an organic acid such as, for example, acetate, maleate, fumarate, citrate, oxalate, succinate, tartrate, malate, mandelate, trifluoroacetate, methanesulphonate and p-toluenesulphonate. A⁻ is preferably an anion selected from chloride, bromide, iodide, sulphate, nitrate, hexafluorophosphate, tetrafluoroborate, acetate, maleate, oxalate, succinate or trifluoroacetate. More preferably A⁻ is chloride, bromide, hexafluorophosphate, tetrafluoroborate, trifluoroacetate or methanesulphonate. Even more preferably, A⁻ is chloride.

The positively charged quaternary amino groups, when present in a sidechain, may be directly linked to the polymer backbone or may be linked via a linker L. L is usually chosen from oligo and poly(alkylene glycols) and alkylene sulphides, short peptide sequences of, for example, 1 to 20 amino acids), alkyl groups, for example C₁-C₆ alkyl groups and short polyester or polycarbonate chains, for example polyester or polycarbonate chains having from 10 to 30 carbon atoms. L is preferably hydrophilic. L may optionally comprise one or more, typically one, cleavable group. The cleavable group is generally a reducible group, for example, —S—S— or an acid cleavable group, for example an acetal group.

The one or more reactive groups are typically present in sidechains attached to the backbone.

Polymers comprising one or more positively charged quaternary amino groups of formula Ia are preferred.

Usually, R₁, R₂ and R₃ are each independently selected from straight or branched C₁-C₄ alkyl groups, phenyl groups, 5- to 6-membered heteroaryl groups, C₃-C₆ cycloalkyl groups, and C₅-C₆ heterocyclyl groups, which C₁-C₄ alkyl groups, phenyl groups, 5- to 6-membered heteroaryl groups, C₃-C₆ cycloalkyl groups, and C₅-C₆ heterocyclyl groups are unsubstituted or substituted with 1 or 2 substituents selected from halogen atoms, hydroxy groups, —CN groups, —NH₂ groups, and —NO₂ groups.

Preferably, R₁, R₂ and R₃ are each independently selected from straight or branched C₁-C₄ alkyl groups and phenyl groups, which C₁-C₄ alkyl groups and phenyl groups are unsubstituted or substituted with 1 substituent selected from halogen atoms and hydroxy groups.

More preferably, R₁, R₂ and R₃ are each independently selected from straight or branched C₁-C₂ alkyl groups, which C₁-C₂ alkyl groups are unsubstituted or substituted with 1 substituent selected from halogen atoms and hydroxy groups.

Even more preferably, R₁, R₂ and R₃ are the same and each represent unsubstituted methyl groups.

Typically, the polymer backbone is based on monomer units (M) chosen from (meth)acrylates, (meth)acrylamides), styryl monomers, vinyl monomers, vinyl ether monomers, vinyl ester monomers, sialic acid monomers, mannose monomers, N-(2-hydroxyethyl)-1-glutamine (HEG) monomers, and ethyleneglycol-oligopeptide monomers. Preferably, the polymer backbone is based on monomer units chosen from N-2-hydroxypropylmethacrylamide (HPMA), N-(2-hydroxyethyl)-1-glutamine (HEG), and ethyleneglycol-oligopeptide, or is a polysialic acid or polymannan polymer.

Polymer backbones based on HPMA are more preferred.

Thus, the polymer typically comprises one or more units of formula

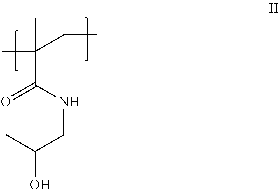

When the polymer backbone is based on HPMA monomer units, and the one or more positively charged quaternary amino groups are of formula Ia, the polymer typically comprises one or more units of formula IIIa and/or IIIb:

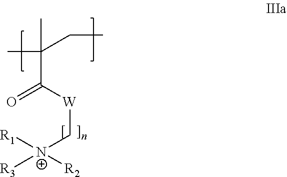

wherein W is S, NH or O, n is an integer from 1 to 4, and R₁, R₂, and R₃ are as defined above;

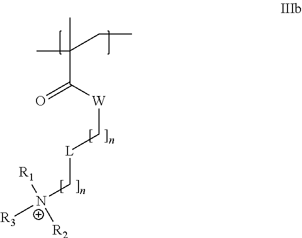

wherein L is a degradable or biodegradable linkage as defined above and W, n, R₁, R₂, and R₃ are as defined above.
W is preferably NH or O, more preferably O.
n is preferably an integer from 1 to 2, more preferably 2.

In the above formulae IIIa and IIIb, R₁, R₂ and R₃ are preferably the same and each represents an unsubstituted methyl group. The positive charges on the nitrogen atoms in formulae IIIa and IIIb are generally associated with a suitable counterion as defined above.

In the above formula lab, L is typically —N—N— or —S—S—, preferably —S—S—.

Units of formula IIIa are preferred.

Usually, the reactive group is a group that will react with a group, e.g. an amino group, present on the surface of a nucleic acid vector. Suitable reactive groups that will react with an amino group include p-nitrophenol (ONp) esters, N-hydroxysuccinimide (NHS) esters and thiazolidine-2-thione groups. Thiazolidine-2-thione groups are preferred.

When the polymer backbone is based on HPMA, and the reactive group comprises a thiazolidine-2-thione group, the polymer typically comprises one or more units of formula IVa and/or IVb:

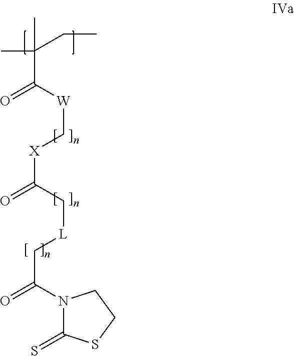

wherein X is O, S or NH, and L and n are as defined above;

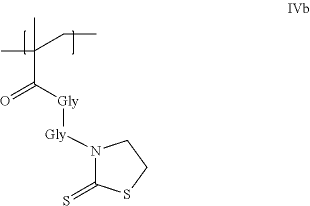

wherein Gly is the amino acid Glycine.
X is preferably NH.

Units of formula IVa are preferred.

In one embodiment, the polymer further comprises one or more biologically active agents as defined above. Said one or more biologically active agents are generally present in sidechains attached to the backbone.

When the polymer backbone is based on HPMA, and the polymer further comprises one or more biologically active agents, the polymer typically includes one or more units of formula V:

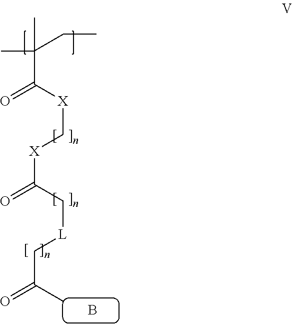

wherein

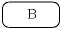

is a biologically active agent as defined herein and X, L and n are as defined above.

In the above formula V,

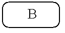

is preferably Epidermal Growth Factor (EGF).

Preferably, the polymer comprises from above 0 to 10 mol % of units of formula Iaa and/or IIIb, preferably IIIa, from above 0 to 14 mol % of units of formula IVa and/or IVb, preferably IVa, and from 0 to 20 mol % of units of formula V, the remaining mol % being generally comprised of units of formula II.

More preferably, the amount of units of formula IIIc and/or IIIb is preferably from 0.25 to 10 mol %, more preferably from 0.5 to 7.5 mol %, even more preferably from 1.5 to 5 mol %.

More preferably, the amount of units of formula IVa or IVb is preferably from 0.5 to 10 mol %, more preferably from 1 to 6 mol %, even more preferably from 2 to 5 mol %.

Even more preferably, in the above formula IIIa or IIIb, n is 2, R₁, R₂ and R₃ are the same and each represents an unsubstituted methyl group and X⁻ is chloride, in the above formula Iva or IVb, X is NH, n is 2 and L is —S—S—, and in the above formula V, X is NH, n is 2, L is —S—S—, and B is EGF.

In a further preferred embodiment of the invention, the polymer comprises a backbone and a side chain, the polymer backbone is based on HPMA, the one or more positively charged quaternary amino groups are of formula Ia and are present in sidechains attached to the backbone, R₁, R₂ and R₃ are the same and each represents an unsubstituted methyl group.

Polymers of the invention can be prepared by analogy with known methods, for example as described in Konak, et al, Langmuir, 2008, 24, 7092-7098, the entirety of which is incorporated herein by reference.

Thus, polymers of the invention are typically prepared by copolymerising one or more monomer units, for example one or more N-2-hydroxypropylmethacrylamide (HPMA), N-(2-hydroxyethyl)-1-glutamine (HEG), ethyleneglycol-oligopeptide, sialic acid or mannose monomer units, preferably HPMA monomer units together with one or more functionalised monomer units comprising positively charged quaternary amino groups together with one or more functionalised monomer units comprising reactive groups, together with, optionally, one or more functionalised monomer units comprising biologically active agents.

Typically, polymers of the invention are prepared by copolymerising one or more monomer units M, as defined herein, with one or more monomer units M, which have been functionalised with reactive groups, with one or more monomer units M-L-N⁺R₁R₂R₃, wherein M, L, R₁, R₂ and R₃ are as defined herein.

Alternatively, the polymers may be prepared by polymerising one or more monomer units, as defined herein, and functionalising the thus-obtained polymer with one or more positively charged quaternary amino groups and one or more reactive groups and, optionally, one or more biologically active agents.

Thus, in the case where the polymer backbone is based on HPMA, polymers of the present invention may be prepared by polymerising one or more units of formula one or more units of formula III′a and/or III′b preferably III′a, one or more units of formula IV′a and/or IV′b, preferably III′a and, optionally, one or more units of formula V′:

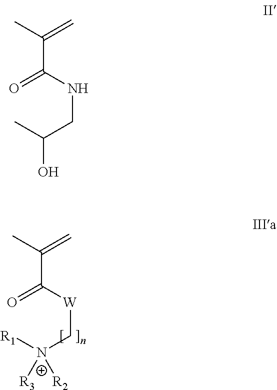

wherein W, n, R₁, R₂, and R₃ are as defined above;

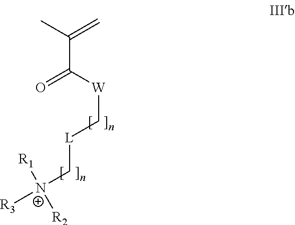

wherein W, n, L, R₁, R₂, and R₃ are as defined above;

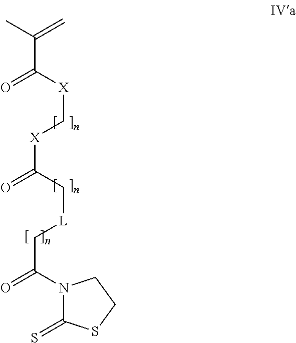

wherein X, n, and L are as defined above;

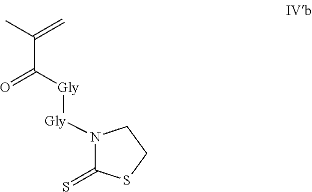

wherein Gly is the amino acid Glycine;

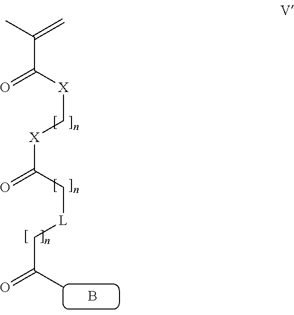

wherein X, n, L and B are as defined above.

The preferred amounts of the monomers II′, III′a, III′b, IV′a, IV′b and V′ used in the copolymerisation reaction are generally the same as the preferred amounts of the units II, IIIa, IIIb, IVa, IVb, and V as defined above.

Typically an initiator is used in the said copolymerisation reaction, preferably AIBN. The reaction generally takes place in an organic solvent, typically DMSO. The reaction is usually heated to a temperature of from 50 to 70° C., preferably about 60° C. The reaction is usually heated to the above-specified temperature for from 4 to 8 hours, preferably 5 to 7 hours, more preferably about 6 hours. The thus-obtained polymers are typically precipitated in an acetone-diethyl ether (3:1) mixture, filtered off, washed with acetone and diethyl ether and dried in vacuo. The thus-obtained polymers may be further purified in Sephadex-LH 20 columns using methanol.

The monomer for the polymerisation reaction are typically commercially available or may be prepared by analogy with known methods, for example as described in Konak, et al, Langmuir, 2008, 24, 7092-7098.

### EXAMPLES

### Example 1

### Protection of Virus Particles from Antibody Interactions Determined by ELISA

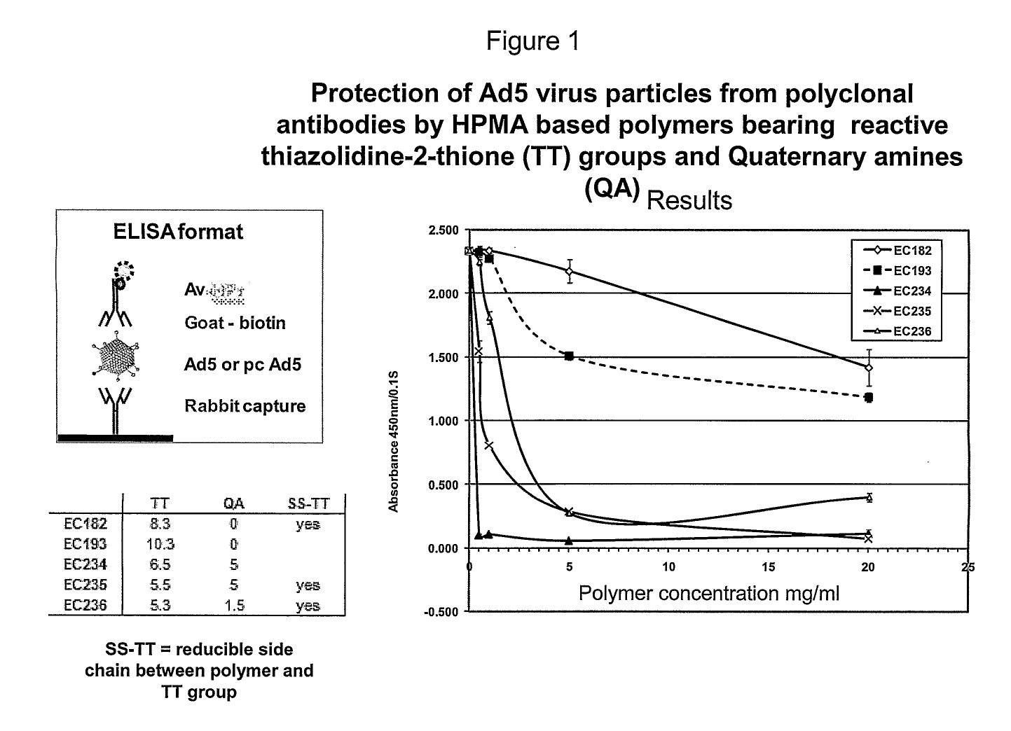

Adenovirus particles (Ad5 wild type) were coated with different concentrations of polymers bearing a range of quaternary amines (QA), and reactive thiazolidine-2-thione (TT) groups (see FIG. 1). Prior to polymer modification, virus particles should be highly purified and free of contaminants that could compete for the TT groups. In this example, virus particles were double banded on caesium chloride gradients following treatment with a Benzonase® (suitable protocols are reported in the literature). Alternatively purification by ion exchange or size exclusion chromatography would also be suitable. Following purification, virus particles were dialysed into reaction buffer (150 mM NaCl, 50 mM HEPES pH 7.8, 2 mM CaCl₂ and MgCl₂). Virus particles were coated in reaction buffer at 20° C. for 1 hour and then placed at 4° C. overnight. Polymer coated virus particles were then separated from unreactive polymers by spin column purification using 5400 columns (Pharmacia). The ability of polyclonal antibodies to bind virus particles was determined by capture ELISA. In this example, ELISA plates were coated with a polyclonal rabbit antibody against Ad5. After blocking and washing, coated virus particles were added at 1e9 particles per well for one hour. Detection was carried out using a biotinylated goat polyclonal antibody with an avidin horse radish peroxidase conjugate secondary. The data show that increasing polymer coating concentration improves protection against antibodies. In addition polymers bearing quaternary amine groups provided greater protection at lower concentrations.

### Example 2

### Influence of Quaternary Amines on the Ability of Polymers to Block Virus Infection of Permissive Cells

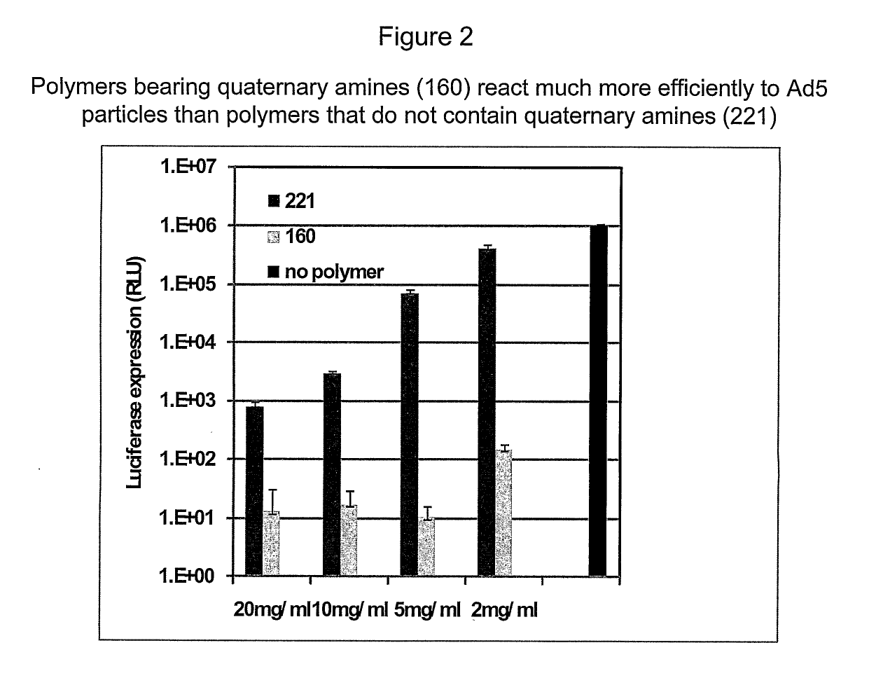

Adenovirus type 5 particles expressing luciferase under the control of the cmv promoter in place of E1 were coated (as above) with polymers containing either 0%, or 7.8% quaternary amines (EC221 and EC160 respectively). The ability of coated virus particle to infect cells was evaluated on A549 (lung carcinoma) monolayers in vitro (FIG. 2). In brief, virus particles (1000 particles per cell) were added to A549 cells growing in a 96-well plate (50,000 cells per well) for 1.5 hours. After a further 24 hours, cells were lysed and luciferase expression was evaluated by luminometry. Polymer coating without retargeting ablates natural virus tropism by preventing the virus from accessing cell surface receptors. The addition of quaternary amines (EC221) enables tropism ablation to occur more efficiently and at much lower concentrations.

### Example 3

### Quaternary Amine Bearing Polymers Protect Ad5 from Interactions with Blood Cells and Enable Infection in the Presence of High Titre Neutralising Serum

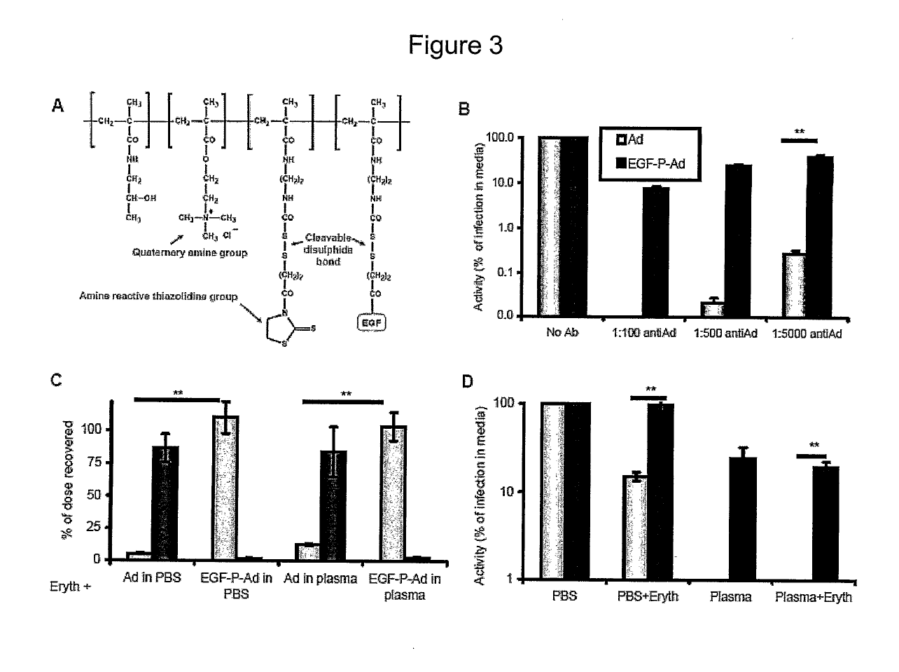

The polymer used in these studies contained quaternary amines and were retargeted with epidermal growth factor (EGF) conjugated to the polymer through the N-terminus (FIG. 3A). A comparison of normal and EGF-mediated infection in neutralising plasma is shown in FIG. 4B. In brief, Ad5 or EGF-P-Ad5 were incubated with dilutions of neutralising antisera and then added to a monolayer of A431 cells, after 90 minutes media was removed and washing performed in PBS and after 24 hours luciferase expression analysed. Note this individual has extremely high titres of neutralising antibodies (1:20,000) against adenovirus relative to the average individual. FIG. 3C Shows that coated virus particles avoid interactions with erythrocytes suspended in PBS/1% BSA or whole fresh human plasma. After incubation, erythrocyte and liquid fractions were separated and assayed for Ad5 genome by quantitative PCR (white=liquid fraction, grey=cell fraction).

FIG. 3D shows a comparison of normal and EGF-mediated infection in presence of human erythrocytes. A431 cells were infected with Ad5 or EGF-P-Ad5 in the presence of a 1 in 5 dilution of erythrocytes suspended in PBS or plasma. After 90 min, media was removed and thorough washing in PBS performed and 24 hours later luciferase expression analysed. Black bars=Ad5, white bars=EGF-P-Ad. N=4, SEM shown, ** p<0.005.

1. A polymer modified virus or viral particle containing therapeutic genetic material or intrinsic therapeutic activity covalently linked to a polymer, which polymer comprises one or more positively charged quaternary amino groups, wherein linkage of the polymer to the virus or viral particle and modification of the same results in inhibition of the virus or viral particle to interact in a host biological system with other molecules with which it would normally interact.

2-6. (canceled)

7. A polymer modified virus or viral particle according to claim 1, wherein the virus or viral particle is based on adenovirus, herpes virus, parvovirus, poxvirus, Togavirus, Rotavirus or picornavirus.

8. A polymer modified virus or viral particle according to claim 1, wherein the virus or viral particle is formed by self assembly between a nucleic acid and a positively charged polymer and/or lipid.

9-11. (canceled)

12. A polymer modified virus or viral particle according to claim 1, wherein the polymer is linked to the virus or viral particle by at least two linkages, typically by at least three linkages.

13. A polymer modified virus or viral particle according to claim 1, wherein the polymer is a multivalent polymer having a polymer backbone based upon monomer units such as N-2-hydroxypropylmethacrylamide (HPMA), N-(2-hydroxyethyl)-1-glutamine (HEG), ethyleneglycol-oligopeptide, or is a polysialic acid or polymannan polymer.

14. A polymer modified virus or viral particle according to claim 1, wherein the polymer and/or the linkages between it and the virus or viral particle are hydrolytically or enzymatically degradable.

15. A polymer modified virus or viral particle according to claim 1, wherein the polymer used to modify the virus or viral particle is crosslinked such that it forms a hydrogel.

16. A polymer modified virus or viral particle as claimed in claim 1, wherein each of the positively charged quaternary amino groups is connected to the polymer backbone either directly or via a spacer group.

17. A polymer modified virus or viral particle according to claim 8, wherein each of the positively charged quaternary amino groups is linked to the polymer backbone via one or more degradable or biodegradable linkages comprising a disulfide bond, a hydrazide bond, an acetal moiety or a bond that is enzymatically cleavable.

18. (canceled)

19. A polymer modified virus or viral particle according to claim 1, wherein a biologically active agent is coupled to or included in the polymer.

20. (canceled)

21. A polymer modified virus or viral particle according to claim 19, wherein the biologically active agent is an antibody or antibody fragment.

22-29. (canceled)

30. A composition comprising a polymer-modified virus or viral particle as defined in claim 1 in association with a suitable diluent or carrier.

31. (canceled)

32. Use of a polymer-modified virus or viral particle as defined in claim 1 in the manufacture of a medicament for use in vaccination or gene therapy, wherein the polymer-modified virus or viral particle comprises therapeutic genetic material.

33. A process for modifying the biological and/or physicochemical properties of a virus or a viral particle, said method comprising reacting said virus or viral particle with a polymer; which polymer comprises one or more positively charged quaternary amino groups and one or more reactive groups, so that the virus or viral particle is linked to the polymer by one or more covalent linkages to obtain a polymer modified virus or viral particle, wherein each of the one or more positively charged quaternary amino groups is connected to the polymer backbone via one or more degradable or biodegradable linkages typically containing reducible or hydrolysable bonds, for example a disulfide bond, a hydrazide bond, an acetal moiety or a bond that is enzymatically cleavable.

34. A polymer modified virus or viral particle according to claim 1, wherein the number of positively charged quaternary amino groups in the polymer is such as to provide from 0.25 mol % to 10 mol % of positively charged quaternary amino groups based on total weight of the polymer.

35. A polymer modified virus or viral particle according to claim 1, wherein the positively charged quaternary amino groups in the polymer have a formula (Ia): 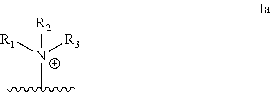
wherein R₁, R₂ and R₃ are each independently selected from the group consisting of straight or branched C₁-C₆ alkyl groups, straight or branched C₂-C₆ alkenyl groups, straight or branched C₂-C₆ alkynyl groups, 6- to 10-membered aryl groups, 5- to 10-membered heteroaryl groups C₃-C₈ cycloalkyl groups and 3- to 8-membered heterocyclyl groups; which C₁-C₆ alkyl groups, C₂-C₆ alkenyl groups, C₂-C₆ alkynyl groups, 6- to 10-membered aryl groups, 5- to 10-membered heteroaryl groups, C₃-C₈ cycloalkyl groups and 3- to 8-membered heterocyclyl groups are unsubstituted or substituted with 1, 2 or 3 substituents selected from the group consisting of halogen atoms, —CN groups, —NH₂ groups, hydroxyl groups, —COOH groups, —NO₂ groups, straight or branched unsubstituted C₁-C₄ alkyl groups, straight of branched C₁-C₄ alkoxy, straight of branched C₁-C₄ alkylthio groups, straight or branched chain C₁-C₄ alkylamino groups, 6- to 10-membered aryloxy groups and phenyl groups; which phenyl groups are typically unsubstituted or substituted with 1, 2 or 3 substituents selected from the group consisting of halogen atoms, —CN groups, —NH₂ groups, hydroxy groups, and —NO₂ groups.

36. A polymer modified virus or viral particle according to claim 1, wherein the positively charged quaternary amino groups in the polymer have a formula (Ib): 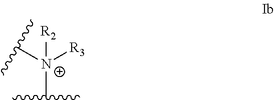
wherein R₂ and R₃ are defined above for compounds of formula (Ia).

37. A polymer modified virus or viral particle, according to claim 1, wherein the positively charged quaternary amino groups in the polymer have a formula (Ic): 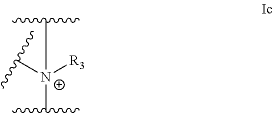
wherein R₃ is defined above for compounds of formula (Ia).

38. A polymer modified virus or viral particle according to claim 1, wherein the positively charged quaternary amino groups in the polymer have a formula (Id): 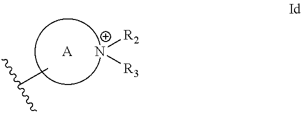
wherein A is a 3 to 8 membered heterocyclyl comprising a nitrogen atom and R₂ and R₃ are defined above for compounds of formula (Ia).

39. A polymer modified virus or viral particle according to claim 1, wherein the positively charged quaternary amino groups in the polymer have a formula (Ie): 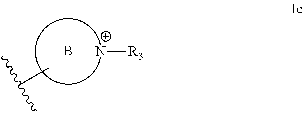
wherein B is a 6- to 10-membered heteroaryl ring comprising a nitrogen atom to which R₃ bonded and wherein R₃ is defined above for compounds of formula (Ia).
